(19) United States
(12) Patent Application Publication
(10) Pub. No.: US 2011/0142964 A1
(43) Pub. Date: Jun. 16, 2011

(54) Increasing athletic performance in humans with ruta chalepensis extract

(76) Inventors: Shawn Wells, Monroe, NC (US); William C. Llewellyn, Palm Beach Gardens, FL (US)

(21) Appl. No.: 12/928,298

(22) Filed: Dec. 8, 2010

Related U.S. Application Data
(60) Provisional application No. 61/283,954, filed on Dec. 10, 2009.

(51) Int. Cl. A61K 36/75; A61P 29/00; A23L 1/30

(52) U.S. Cl. 424/725; 426/2; 426/648

(57) ABSTRACT

The present invention relates to increasing the athletic performance of humans, using ruta chalepensis extract as an oral supplement or medicine. Use of the invention provides increased muscle strength, muscle power, endurance, muscle protein content, and/or reduced fatigue following exercise or competition.

### CROSS-REFERENCE TO RELATED APPLICATIONS

This patent application claims the benefit of provisional patent application 61283954, filed on Dec. 10,2009.

### STATEMENT REGARDING FEDERALLY SPONSORED RESEARCH OR DEVELOPMENT

Not Applicable

### REFERENCE TO A MICROFICHE APPENDIX

Not Applicable

### BACKGROUND OF THE INVENTION

In recent years there has been growing interest in identifying natural medicines and plant extracts that can be used to improve athletic performance or body composition (the ratio of lean to fat mass) in humans. Traditional Chinese and Ayurvedic healing practices have been good sources for identifying plants of notable medicinal value, as many have been used for centuries to treat disease. In an isolated number of instances, traditional medicine protocols and extracts have been further investigated and modified by modern scientists, and made useful for improving the performance of athletes. Given the limited progress in this area, however, these inventors set out to further explore, identify, and develop strategies for more effective herbal substances for improving athletic performance.

Ruta chalepensis is a plant with a long history of use in both Ayurvedic and Chinese traditional medicine practices. It is still widely used today to treat pain and reduce fever, and is also known to possess several other distinct medicinal qualities. In spite of this wide use, however, ruta chalepensis has not been explored as a potential medicine or health food supplement for improving athletic performance in animals or humans. After extensive research and investigation, these inventors have developed a novel use for ruta chalepensis extract. When properly applied, this extract improves muscle protein content and athletic performance in human athletes and other active exercising individuals.

### BRIEF SUMMARY OF THE INVENTION

The present invention involves extracts of the ruta chalepensis plant for improving athletic performance in humans. The ruta chalepensis extract is given orally to an athlete or exercising individual, in capsule, pill, powder, edible bar, or liquid form. The extract is given in an oral dose ranging from between 100 mg and 5,000 mg. This dose is repeated daily, or on a regular schedule consistent with training (such as in conjunction with training days if daily use is not preferred). For most notable results, the extract is given for a minimum period of 2-3 weeks. When taken consistently in such a manner, and when its use is accompanied by regular athletic competition, exercise, or high physical activity, ruta chalepensis extract can significantly improve athletic performance. This is seen by improvements in such variables as running speed, endurance, muscle strength, and/or muscle protein content. Such improvements may be noticed either during athletic competition, or during/following exercise in support of such activity. In addition, the ratio of lean muscle mass to fat mass may be increased in the body as a result of supplementing with ruta chalepensis extract.

### BRIEF DESCRIPTION OF THE SEVERAL VIEWS OF THE DRAWING

Not Applicable

### DETAILED DESCRIPTION OF THE INVENTION

Ruta chalepensis is a perennial herbal of the Rutaceae family. Most commonly identified as “rue”or “ruda”, this plant is found in many regions of the world, including North and South America, Europe, and Asia. Ruta chalepensis is considered a medicinal plant, and has a long history of use to treat disease in Ayurvedic and Chinese traditional medicine. Its specific uses in these areas of natural medicine have been many (J Ethnopharmacol 28 (1960) pp. 305-312). In Saudi Arabia, for example, ruta chalepensis extract is used as an analgesic, anti-inflammatory, and fever reducing medicine. In India, it is applied for the treatment of edema, fever, and pain, and also to treat certain bleeding disorders. In China, ruta chalepensis extracts are commonly used to treat convulsions and certain nervous disorders in children. They are also used to counter the toxic effects of certain types of venom. In Africa, the plant extracts are also often used to treat fever.

While it may be well understood that ruta chalepensis extract possesses a spectrum of medicinal/biologically active properties, a full understanding of these medicinal/biologically active properties and their underlying mechanisms of action is lacking. Chemical analysis of the plant has yielded a wide variety of potentially active molecules. More than two dozen molecules have been recovered during screening of the plant, including alkaloids, flavonoids, coumarins, tannins, volatile oil, volatile bases, cardiac glycosides, glucosinolates, anthraquinones, sterols, and triterpenes (J Pharm Sci 55 (1966), 225-272). These classes of natural compounds are known to include a great many members with strong biological activity in animals. Given such a broad range of action for this medicinal herb, it seems logical to conclude that in many cases, an obvious inherent medicinal property may actually be the result of more than one chemical actor, and in some cases may actually involve the cooperation of several active molecules.

Of the known medicinal activities of the ruta chalepensis plant, its effects as an analgesic and fever reducing treatment are among the best documented (Nat Prod Res 19(3) 2005, pp. 203-210). Studies point to an inherent anti-inflammatory action as the root of both activities. In this context, active molecules of the ruta chalepensis plant interfere with the biosynthesis of endogenous prostaglandins, which are strong mediators of inflammation, pain, and fever in humans and other animals. Extract of ruta chalepensis was also shown to increase androgen (testosterone) levels in male mice by 365-476% during one study (J Appl Res 5(1), 2005 pp. 206-11). This also supports the notion that the plant has noteworthy hormone-modulating activity. Additional studies in mature rats have demonstrated the plant has insulin stimulating action. During one study, ruta chalepensis extract was shown to increase insulin levels by 180%, which corresponded to a 40-50% increase in liver, heart, and skeletal muscle glycogen levels (J Appl Res 4(4), 2004 pp. 625-29).

Prior to the work of these inventors, no investigations into the effects of ruta chalepensis extract on athletic performance or muscle protein content in humans or animals have taken place. There has also been no unsupported suggestion or theory in the medical literature that ergogenic activity may be present in this plant, nor any mention of such attempted use. The use of this plant according to this invention came only after a long effort to research Ayurvedic and traditional Chinese medical preparations, and discover what, and under what context, particular plant species can be used to improve the results of exercise and athletic performance. Ruta chalepensis was shown to possess unique beneficial activities in this regard, according to these inventors.

It is the object of this invention to improve athletic performance by the oral administration of ruta chalepensis extract. The ruta chalepensis extract used for the purpose of this invention may have been obtained with any of a variety of standard extraction methods. These typically involve the blending of dried plant material with one or more liquid extraction agents. The extraction agent is later separated from the leftover plant material, and then dried, heated, evaporated, distilled, or otherwise removed from the mixture. This will yield a powdered extract that contains medically active plant molecules. The subject of this invention is ruta chalepensis extract obtained with at least one extraction agent selected from the group consisting of water, alcohol, chloroform, petrol ether, dichloromethane, acetic acid, liqueur wine, and a supercritical liquid. When said extraction agent is an alcohol, at least one alcohol selected from the group consisting of methanol, ethanol, propanol, butanol, pentanol and hexanol may be used.

Athletic performance is defined as any sporting activity where muscle strength and/or endurance are integral to individual performance, and is inclusive of both aerobic and anaerobic metabolic conditions. This includes active athletic competition, such as baseball, football, running, or Track & Field. It also includes any exercise activity designed to improve physical performance, directly or indirectly, via an improvement in muscle protein content (size), strength, and/or endurance, such as resistance training (circuit equipment, weight training), aerobics, Pilates, and/or calisthenics. Any improvement in athletic performance must include objective increases in speed, strength, muscle size (protein content), and/or endurance. It may also involve improved recovery from fatigue following exercise or athletic competition.

Surprisingly, it was shown during our studies that ruta chalepensis extract acts as an effective in-vivo peroral agent for improving athletic performance when used according to our recommended protocols. We found this herbal plant extract to possess a wide spectrum of performance-enhancing activity, in fact. This includes an ability to increase muscle strength, muscle power, endurance, and muscle protein content (size), as well as to reduce fatigue and facilitate recovery following intense exercise or athletic competition. The benefits appear almost immediately in some cases, but are more likely to occur after several weeks of regular use of the extract. The benefits were apparent in such level that it appears highly likely that ruta chalepensis extract can have a very strong influence over the exercise and during-competition performance of supplemented athletes.

The present invention provides methods of using pharmaceutical and dietary health supplement compositions of the inventive compound. Such compositions may be for oral administration. The treatment may consist of a single dose or a plurality of doses over a period of time. In general, comprehended by the invention are compositions comprising effective amounts of a compound of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 90), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hyaluronic acid may also be used, and this may have the effect of promoting sustained duration in the circulation. The pharmaceutical and dietary health supplement compositions optionally may include still other pharmaceutically acceptable liquid, semisolid, or solid diluents that serve as pharmaceutical vehicles, excipients, or media, including but are not limited to, polyoxyethylene sorbitan monolaurate, magnesium stearate, methyl- and propylhydroxybenzoate, starches, sucrose, dextrose, gum acacia, calcium phosphate, mineral oil, cocoa butter, and oil of theobroma. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435 1712 which are herein incorporated by reference. The compositions may be prepared in liquid form, or may be in dried powder, such as lyophilized form. Sustained release formulations are also contemplated, as are transdermal formulations.

Contemplated for use herein are oral solid dosage forms, which are described generally in Remington's Pharmaceutical Sciences, 18th Ed. 1990 (Mack Publishing Co. Easton Pa. 18042) at Chapter 89, which is herein incorporated by reference. Solid dosage forms include tablets, capsules, pills, troches or lozenges, cachets or pellets, and powder that may or may not be dissolved in a liquid. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the ruta chalepensis extract is illustrated by Marshall, K., Modern Pharmaceutics, Edited by G. S. Banker and C. T. Rhodes Chapter 10, 1979, herein incorporated by reference. In general, the formulation will include the inventive compound, and inert ingredients which allow for protection against the stomach environment, and release of the biologically active material in the intestine. The oral dosage forms contemplated herein can be administered at any time of the day or night. These oral dosage forms may be administered or taken one or more times per day or night. It is preferable that the oral dosage forms be administered or taken between 1 and 10 times per day or night. It is still more preferable that the oral dosage forms be administered or taken between 1 and 5 times per day or night. It is still more preferable that the oral dosage forms be administered or taken between 2 and 3 times per day or night.

Also specifically contemplated are oral dosage forms of the above inventive compounds. If necessary, the compounds may be chemically modified so that oral delivery is efficacious. Generally, the chemical modification contemplated is the attachment of at least one moiety to an active molecule itself, where said moiety permits (a) inhibition of proteolysis; and (b) uptake into the blood stream from the stomach or intestine. Also desired is the increase in overall stability of the compound and increase in circulation time in the body. Examples of such moieties include: Polyethylene glycol, copolymers of ethylene glycol and propylene glycol, carboxymethyl cellulose, dextran, polyvinyl alcohol, polyvinyl pyrrolidone and polyproline (Abuchowski and Davis, Soluble Polymer-Enzyme Adducts, Enzymes as Drugs, Hocenberg and Roberts, eds., Wiley-Interscience, New York, N.Y., (1981), pp 367 383; Newmark, et al., J. Appl. Biochem. 4:185 189 (1982)). Other polymers that could be used are poly-1,3-dioxolane and poly-1,3,6-tioxocane. Preferred for pharmaceutical usage, as indicated above, are polyethylene glycol moieties.

For the oral delivery dosage forms, it is also possible to use a salt of a modified aliphatic amino acid, such as sodium N-(8-[2-hydroxybenzoyl]amino) caprylate (SNAC), as a carrier to enhance absorption of the compounds of this invention. The clinical efficacy of a heparin formulation using SNAC has been demonstrated in a Phase II trial conducted by Emisphere Technologies. See U.S. Pat. No. 5,792,451, “Oral drug delivery composition and methods”.

The ruta chalepensis extract can be included in the formulation as fine multiparticulates in the form of granules or pellets of particle size about 1 mm. The formulation of the material for capsule administration could also be as a powder, lightly compressed plugs or even as tablets. The compound could be prepared by compression.

Colorants and flavoring agents may all be included. For example, the protein (or derivative) may be formulated (such as by liposome or microsphere encapsulation) and then further contained within an edible product, such as a refrigerated beverage containing colorants and flavoring agents.

One may dilute or increase the volume of the ruta chalepensis extract with an inert material. These diluents could include carbohydrates, especially mannitol, .alpha.-lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of ruta chalepensis extract into a solid dosage form. Materials used as disintegrants include but are not limited to starch including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants is the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the ruta chalepensis extract together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the contemplated compounds.

Controlled release formulation may be desirable. The contemplated compounds could be incorporated into an inert matrix which permits release by either diffusion or leaching mechanisms e.g., gums. Slowly degenerating matrices may also be incorporated into the formulation, e.g., alginates, polysaccharides. Another form of a controlled release of the contemplated compounds is by a method, wherein the compound is enclosed in a semipermeable membrane which allows water to enter and push the compounds out through a single small opening due to osmotic effects. Some enteric coatings also have a delayed release effect.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto The following non-limiting example and data illustrates various aspects and features relating the methods and compositions of the present invention. While the utility of this invention may be illustrated through the use of several methods and compositions, it will be understood by those skilled in the art that comparable results are obtainable with various other methods, as are commensurate with the scope of this invention.

### EXAMPLE I

Effective Dosages

During the course of developing this invention, an effective oral daily dosage of ruta chalepensis extract to improve athletic performance was determined to be between 100 mg to 5,000 mg. The total daily dosage can be further subdivided for more sustained blood concentrations of the extract's constituents, with 2-3 applications per day being most preferred. The preferred embodiment of this invention involves the daily oral use of ruta chalepensis extract, which is continued for a minimum of 2-3 weeks. The supplementation accompanies an athletic competition or exercise program, or active lifestyle with significant physical activity.

We claim:

1. A method of increasing athletic performance in humans, which comprises administering orally an effective amount of ruta chalepensis extract.

2. A composition to be administered orally to humans for increasing athletic performance, which comprises an effective amount of ruta chalepensis extract.

3. A medicine or dietary health supplement comprising the composition of claim 2 in capsule, pill, powder, edible bar, or liquid forms.

4. A composition of claim 2, wherein the ruta chalepensis extract is obtained with at least one component selected from the group consisting of water, alcohol, chloroform, petrol ether, dichloromethane, acetic acid, liqueur wine, and a supercritical liquid as an extraction agent.

5. A composition of claim 4, wherein said extraction agent is at least one alcohol selected from the group consisting of methanol, ethanol, propanol, butanol, pentanol and hexanol.
